(19) United States
(12) Patent Application Publication
(10) Pub. No.: US 2013/0186408 A1
(43) Pub. Date: Jul. 25, 2013

(54) Air Supply Tube For Nasal Breathing Apparatus

(71) Applicant: ERIC CHANG, TAICHUNG CITY (TW)

(72) Inventor: ERIC CHANG, TAICHUNG CITY (TW)

(73) Assignee: HSINER CO, LTD., Taichung City (TW)

(21) Appl. No.: 13/354,451

(22) Filed: Jan. 20, 2012

(51) Int. Cl. A61M 15/08; A61M 16/00

(52) U.S. Cl. 128/207.18

(57) ABSTRACT

An air supply tube for a nasal breathing apparatus is made of a silicone material, and includes a tubular wall surrounding an axis and defining an axial hole. The tubular wall is corrugated, and includes a plurality of annularly-shaped concave and convex wall portions alternately arranged along the length of the air supply tube. The convex wall portions are farther from the axis than the concave wall portions. The concave wall portions have a first radial thickness which is larger than a second radial thickness of the convex wall portion.

### BACKGROUND OF THE INVENTION

1. Field of the Invention

This invention relates to a medical supply, and more particularly to an air supply tube for a nasal breathing apparatus.

2. Description of the Related Art

A currently available nasal breathing apparatus includes a main body, a head strap connected to two opposite sides of the main body, a nose piece connected to an inner side of the main body, and an air supply tube connected to an outer side of the main body opposite to the nose piece. The air supply tube is generally made a plastic material, such as polyethylene (PE), or a silicone material. The air supply tube made of the plastic material is not deformable, that is, it is hard and not bendable. Hence, in the presence of an external force, the breathing apparatus is easily displaced by the air supply tube. This leads to discomfort and dysfunction of the breathing apparatus when in use.

Further, referring to FIG. 1, a conventional air supply tube 1 that is made of the silicone material has a tubular wall 101 that is corrugated and that includes a plurality of annularly-shaped concave and convex wall portions 102, 103 alternately arranged along the length of the air supply tube 1. The concave wall portions 102 have a radial thickness equal to that of the convex wall portions 103.

However, because the silicone-made air supply tube 1 is soft, when use, it easily bends and deforms so that the sectional area of the air supply tube 1 becomes small, thereby leading to change in the amount of flow and speed of air inside the tube 1. This creates a wrong judgment on the breathing frequency of the user, which in turn leads to insufficient supply of air to the user or affecting the sleep quality of the user.

### SUMMARY OF THE INVENTION

Therefore, an object of the present invention is to provide an air supply tube for a nasal breathing apparatus that has good anti-deformation strength and that pan ensure normal supply of air.

According to this invention, an air supply tube for a nasal breathing apparatus is made of a silicone material, and comprises a tubular wall surrounding an axis and defining an axial hole. The tubular wall is corrugated, and includes a plurality of annularly-shaped concave and convex wall portions alternately arranged along the length of the air supply tube. The convex wall portions are farther from the axis than the concave wall portions. The concave wall portions have a first radial thickness which is larger than a second radial thickness of the convex wall portion.

### BRIEF DESCRIPTION OF THE DRAWINGS

Other features and advantages of the present invention will become apparent in the following detailed description of the preferred embodiments with reference to the accompanying drawings, of which:

FIG. 1 is a partly sectional view of a conventional air supply tube;

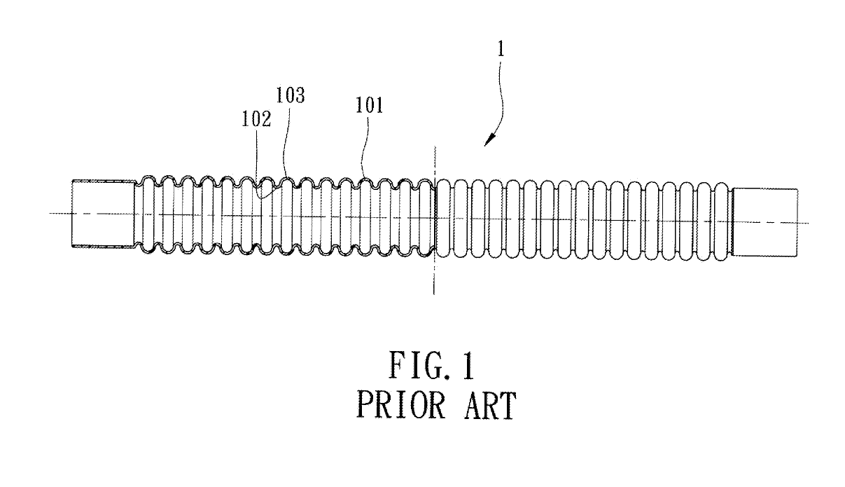

FIG. 2 is a partly sectional view of an air supply tube for a nasal breathing apparatus according to the first preferred embodiment of this invention;

FIG. 3 is an enlarged fragmentary sectional view of FIG. 2;

FIG. 4 is a sectional view of an air supply tube for a nasal breathing apparatus according to the second preferred embodiment of this invention;

FIG. 5 is an enlarged fragmentary sectional view of FIG. 4;

FIG. 6 is a sectional view of an air supply tube for a nasal breathing apparatus according to the third preferred embodiment of this invention;

FIG. 7 is an enlarged fragmentary sectional view of FIG. 6;

FIG. 8 is another fragmentary sectional view of FIG. 6;

FIG. 9 is a sectional view of an air supply tube for a nasal breathing apparatus according to the fourth preferred embodiment of this invention;

FIG. 10 is an enlarged fragmentary sectional view of FIG. 9;

FIG. 11 is a sectional view of an air supply tube for a nasal breathing apparatus according to the fifth preferred embodiment of this invention;

FIG. 12 is an enlarged fragmentary sectional view of a first tube section of the fifth preferred embodiment; and

FIG. 13 is an enlarged fragmentary sectional view of a second tube section of the fifth preferred embodiment.

### DETAILED DESCRIPTION OF THE PREFERRED EMBODIMENTS

The above-mentioned and other technical contents, features, and effects of this invention will be clearly presented from the following detailed description of five preferred embodiments in coordination with the reference drawings.

Before this invention is described in detail, it should be noted that, in the following description, similar elements are designated by the same reference numerals.

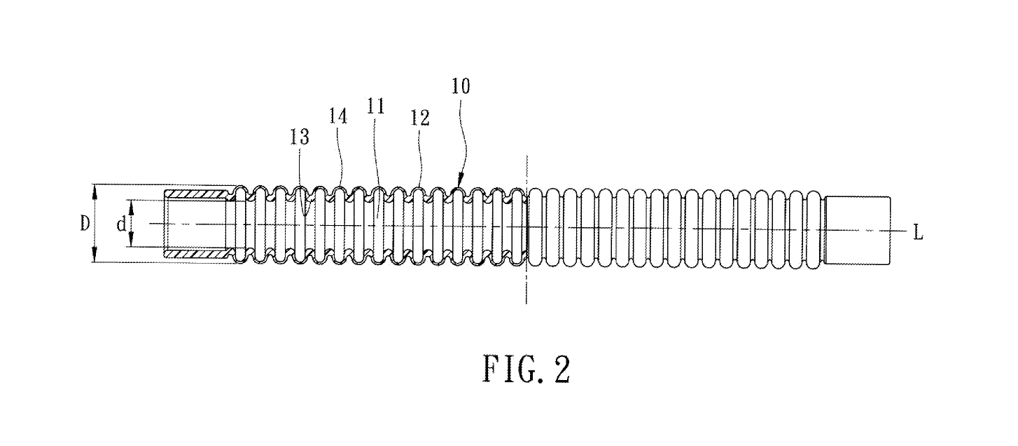

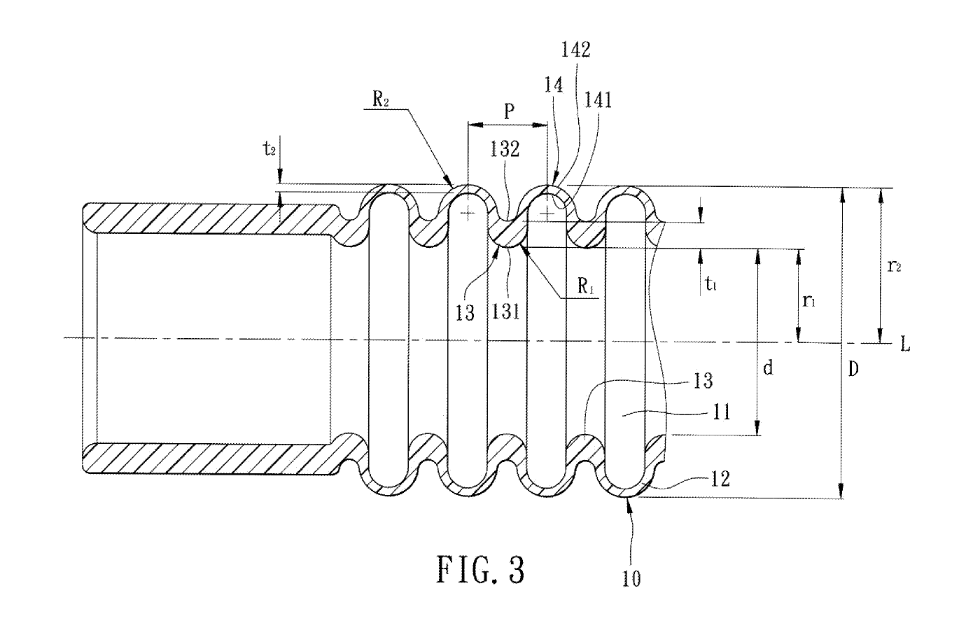

Referring to FIGS. 2 and 3, an air supply tube 10 for a nasal breathing apparatus according to the first preferred embodiment of the present invention is made of a silicone material, and comprises a tubular wall 12 surrounding an axis (L) and defining an axial hole 11. The tubular wall 12 is corrugated, and includes a plurality of annularly-shaped concave and convex wall portions 13, 14 alternately arranged along the length of the air supply tube 10. The convex wall portions 14 are farther from the axis (L) than the concave wall portions 13. The concave wall portions 13 have a first radial thickness (t₁) that is larger than a second radial thickness (t₂) of the convex wall portions 14. The ratio of the first radial thickness (T₁) to the second radial thickness (t₂) is larger than 1, but smaller than 4. In this embodiment, the first radial thickness (t₁) is 1.75 mm, while the second radial thickness (t₂) is 0.55 mm Hence, the ratio of the first radial thickness (t₁) to the second radial thickness (t₂) is 3.18.

Each of the concave wall portions 13 has an inner annular surface 131 adjacent to the axial hole 11, and an outer annular surface 132 opposite to the inner annular surface 131. Each of the convex wall portions 14 has an inner annular surface 141 adjacent to the axial hole 11 and connected between the inner annular surfaces 131 of two adjacent ones of the concave wall portions 13, and an outer annular surface 142 opposite to the inner annular surface 141 and connected between the outer annular surfaces 132 of the two adjacent ones of the concave wall portions 13. A pitch (P) between two adjacent ones of the convex wall portions 14 is in the range of 4˜6 mm. In this embodiment, the pitch (P) is 5.44 mm. The outer annular surface 142 of each convex wall portion 14 has a largest outer diameter (D). The inner annular surface 132 of each concave wall portion 13 has a smallest inner diameter (d). The ratio of the largest outer diameter (D) to the smallest inner diameter (d) is larger than 1.5, but smaller than 2. In this embodiment, the largest outer diameter (D) is 21 mm, while the smallest inner diameter (d) is 12.6 mm. Hence, the ratio of the largest outer diameter (D) to the smallest inner diameter (d) is 1.67. Further, the outer annular surface 142 of each convex wall portion 14 has a largest outer radius (r₂) greater than a smallest inner radius (r₁) of the inner annular surface 131 of each concave wall portion 13 by an amount ranging from 3.5˜4.5 mm. In this embodiment, the largest outer radius (r₂) is greater than the smallest inner radius (r₁) by 4.2 mm. The inner annular surface 131 of each concave wall portion 13 further has a curvature radius (R₁) that is in the range of 1.3˜1.8 mm. In this embodiment, the curvature radius (R₁) of the inner annular surface 131 is 1.35 mm. The outer annular surface 142 of each convex wall portion 14 further has a curvature radius (R₂) that is in the range of 1.5˜2.2 mm. In this embodiment, the curvature radius (R₂) of the outer annular surface 142 is 2.03 mm.

Therefore, because the first radial thickness (t₁) is larger than the second radial thickness (t₂), the concave wall portions 13 are stiffer, while the convex wall portions 14 are more deformable. When the air supply tube 10 bends in the presence of an external force, because the concave wall portions 13 provide strong resistance against deformation, the axial hole 11 can maintain a fixed cross-sectional area, thereby ensuring normal supply of air to the user or maintaining the sleep quality of the user.

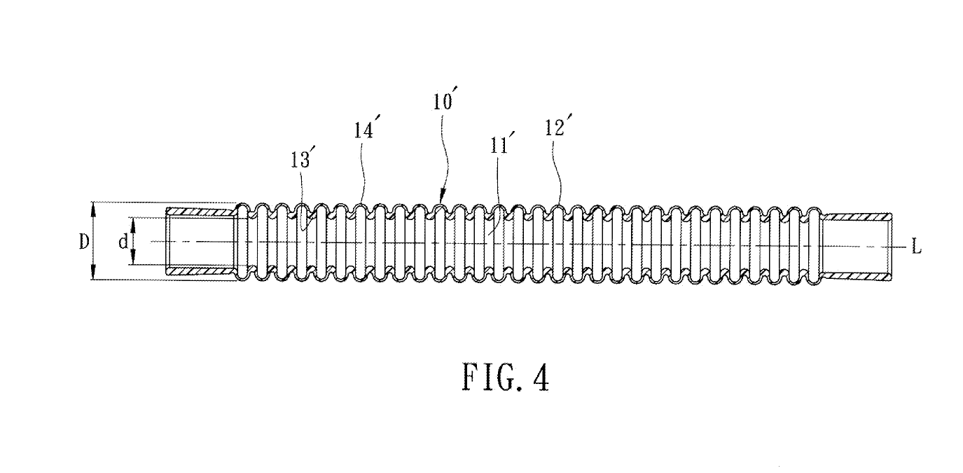

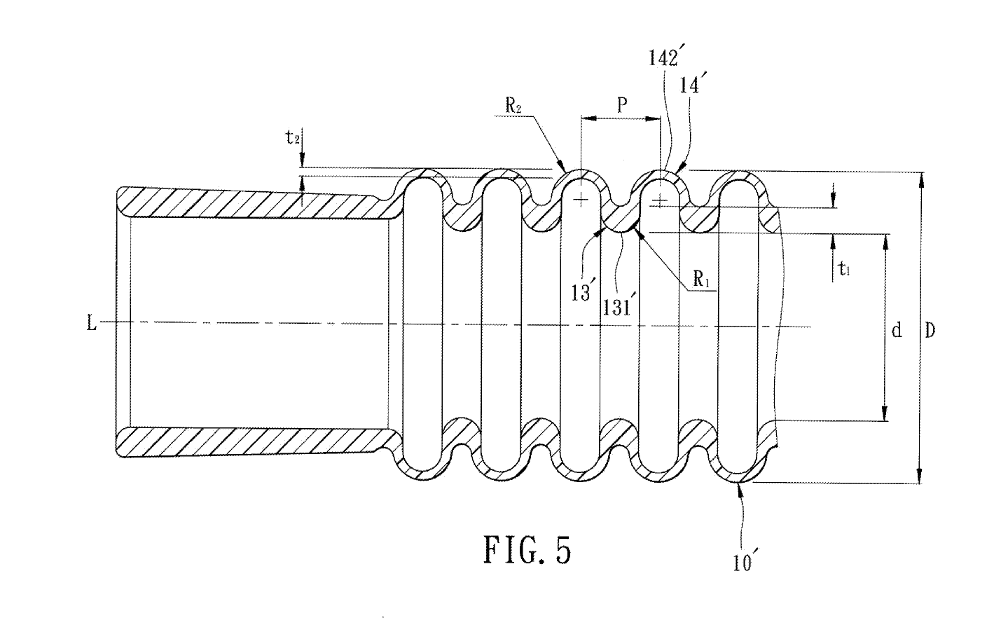

Referring to FIGS. 4 and 5, an air supply tube 10′ for a nasal breathing apparatus according to the second preferred embodiment of this invention is shown to be similar to the first preferred embodiment. However, in this embodiment, the first radial thickness (t₁) is 1.75 mm, while the second radial thickness (t₂) is 0.58 mm. Hence, the ratio of the first radial thickness (t₁) to the second radial thickness (t₂) is 3.02. The purpose and effect of the first preferred embodiment can be similarly achieved using the second preferred embodiment.

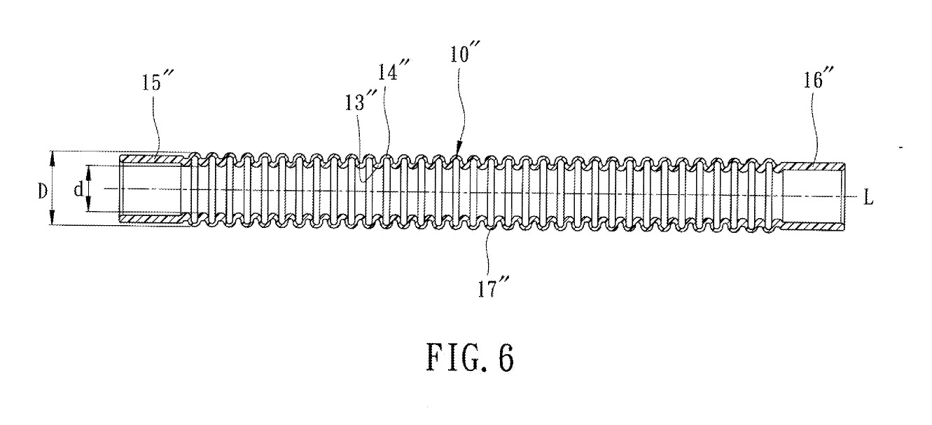

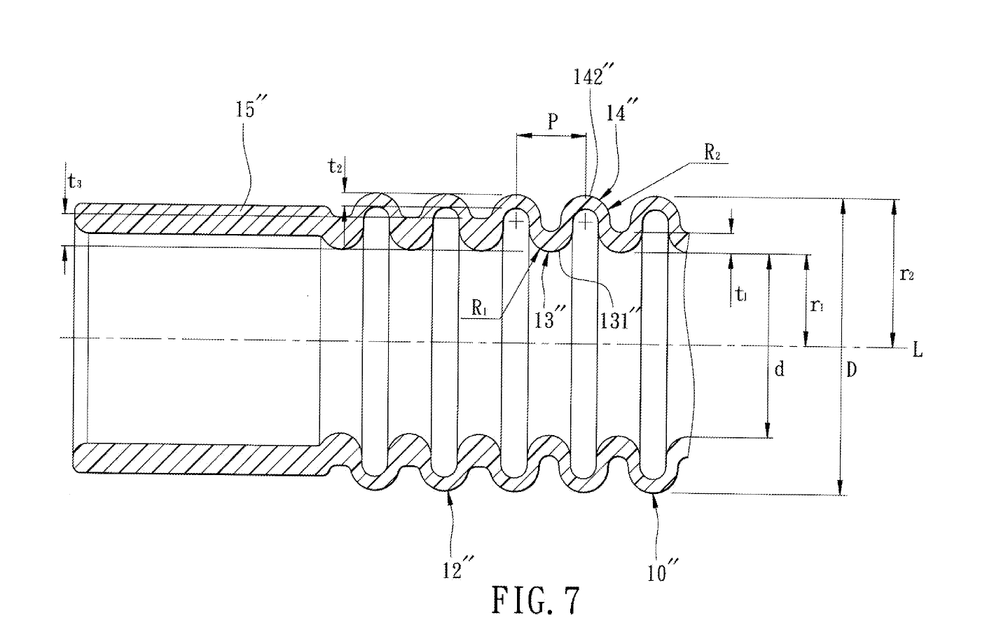

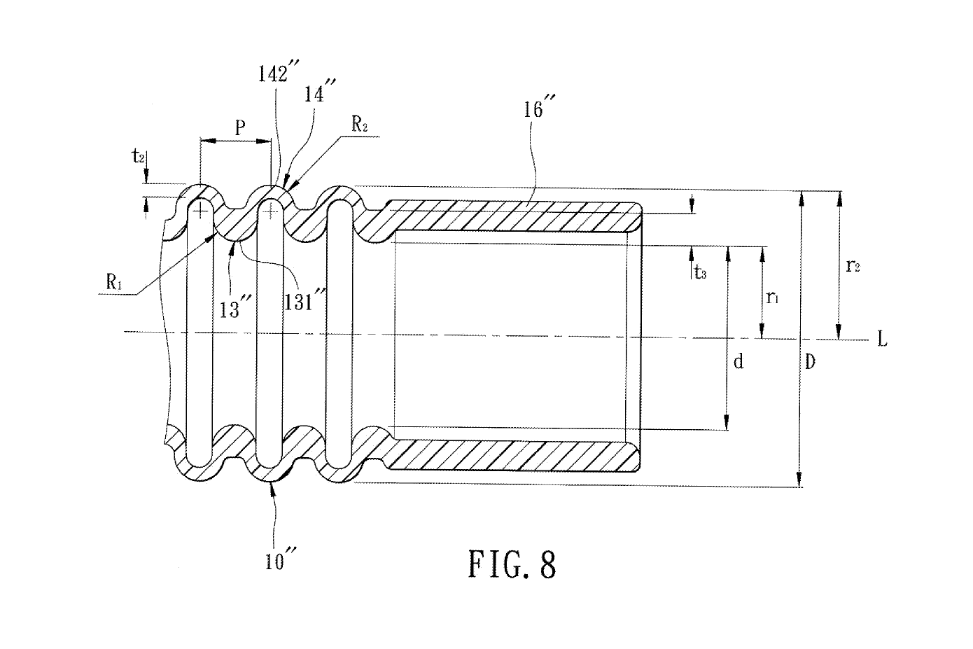

Referring to FIGS. 6 to 8, an air supply tube 10″ for a nasal breathing apparatus according to the third preferred embodiment of the present invention is shown to be similar to the first preferred embodiment. However, in this embodiment, the concave wall portions 13″ at an intermediate tubular portion 17″ of the tubular wall 12″ have a first radial thickness (t₁) of 1.35 mm, and the convex wall portions 14″ at the intermediate tubular portion 17″ is 0.9 mm. The convex wall portions 14″ at first and second tubular ends 15″, 16″ of the tubular wall 12″ that are connected respectively to two opposite sides of the intermediate tubular portion 17″ have a second radial thickness (t₇) similar to that of the intermediate tubular portion 17″. The concave wall portions 13″ at the first and second tubular ends 15″, 16″ have a third radial thickness (t₃) of 2.17 mm. Hence, each of the first radial thickness (t₁) and the third radial thickness (t₃) is larger than the second radial thickness (t₂), and the ratio of the first radial. thickness (t₁) to the second radial thickness (t₂) is 1.5. Further, in this embodiment, the pitch (P) is 4.8 mm, the largest outer diameter (D) is 20 mm, and the smallest inner diameter (d) is 12.4 mm. Moreover, the largest outer radius (r₂) is greater than the smallest inner radius (r₁) by 3.8 mm. Additionally, the curvature radius (R₁) of the inner annular surface 131″ of the concave wall portions 13″ is 1.5 mm, and the curvature radius (R₂) of the outer annular surface 142″ is 1.7 mm. The purpose and effect of the first preferred embodiment can be similarly achieved using the third preferred embodiment.

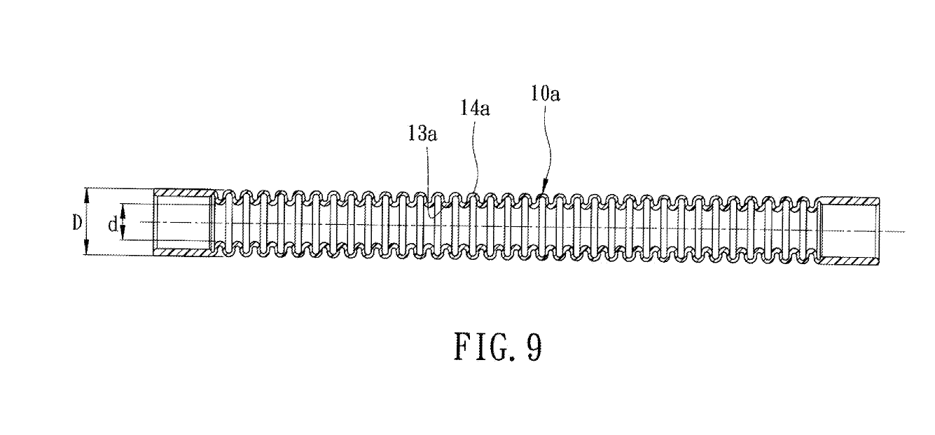

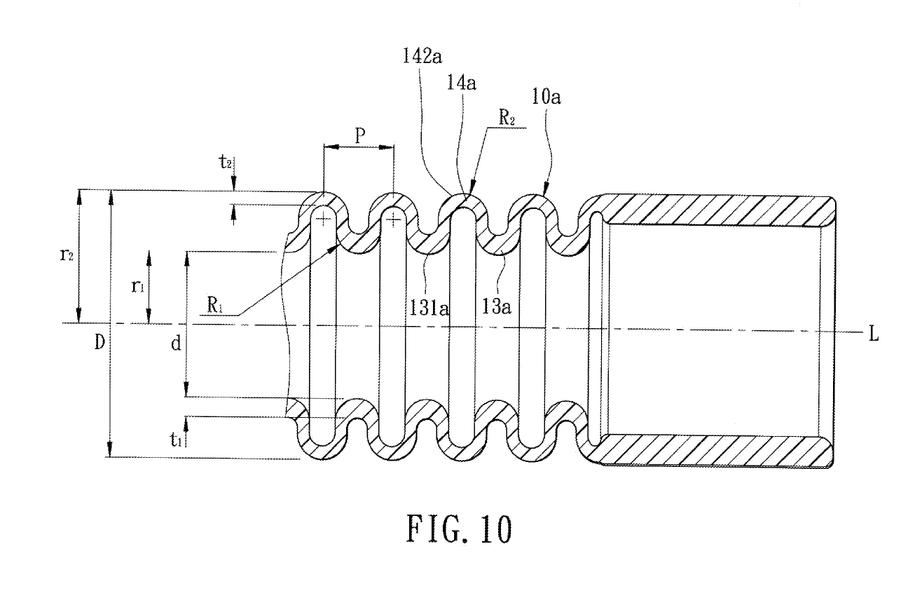

Referring to FIGS. 9 and 10, an air supply tube (10a) for a nasal breathing apparatus according to the fourth preferred embodiment of the present invention is shown to be similar to the first preferred embodiment. However, in this embodiment, the first radial thickness (t₁) is 1.3 mm, the second radial thickness (t₂) is 0.9 mm, and the ratio of the first radial thickness (t₁) to the second radial thickness (t₂) is 1.44. Further, the pitch (P) is 4.8 mm, the largest outer diameter (D) is 18.2 am, the smallest inner diameter (d) is 9.8mm, and the largest outer radius (r₂) is greater than the smallest inner radius (r₁) by 4.1 mm. Moreover, the curvature radius (R₁) of the inner annular surface (131a) is 1.51 mm, and the curvature radius (R₂) of the outer annular surface (142a) is 1.7 mm. The purpose and effect of the first preferred embodiment can be similarly achieved using the fourth preferred embodiment.

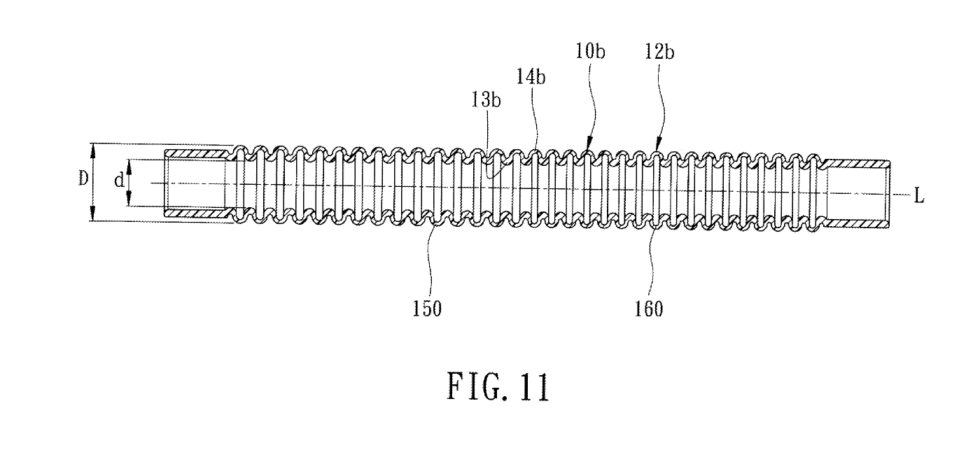

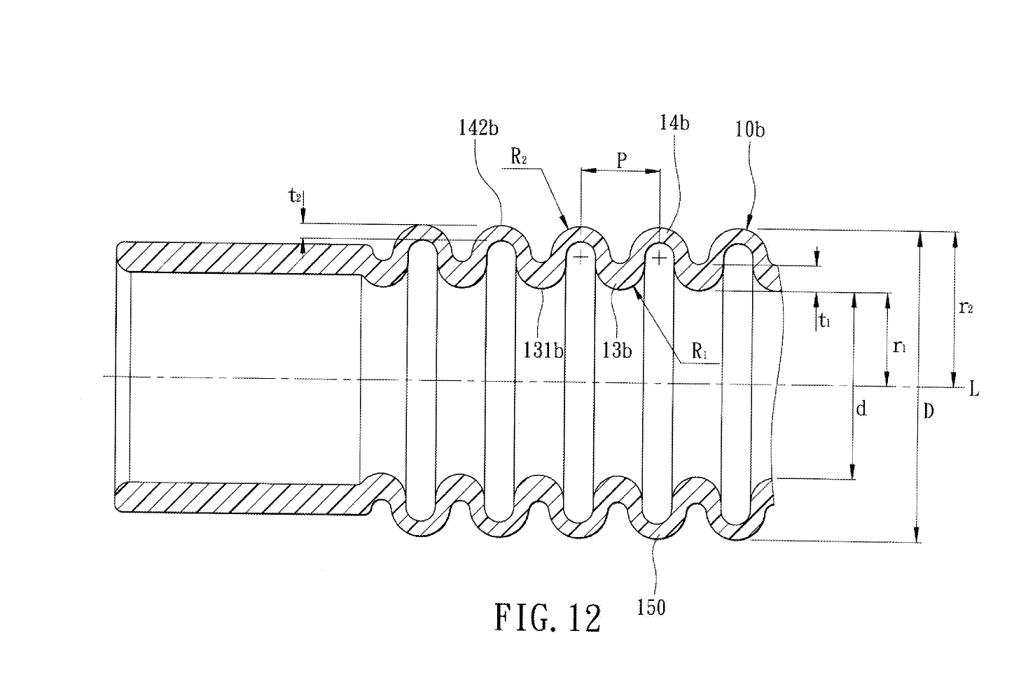

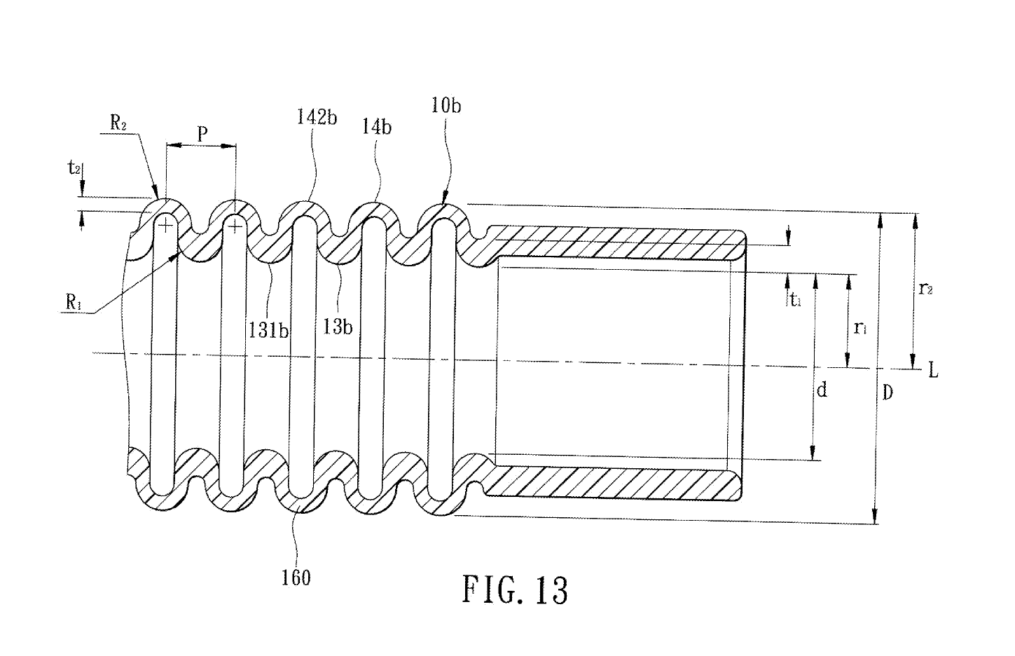

Referring to FIGS. 11 to 13, an air supply tube (10b) for a nasal breathing apparatus according to the fifth preferred embodiment of the present invention is shown to be similar to the first preferred embodiment. However, in this embodiment, in a first tubular section 150 of the tubular wall (12b), the first radial thickness (t₁) of the concave wall portions (13b) is 1.75 mm, the second radial thickness (t₂) of the convex wall portions (14b) is 1.0 mm, and the ratio of the first radial thickness (t₁) to the second radial thickness (t₂) is 1.75. Further, the pitch (P) is 5.44 mm, the curvature radius (R₁) of the inner annular surface (131b) is 1.7 mm, and the curvature radius (R₂) of the outer annular surface (142b) is 2.03 mm. In a second tubular section 160 of the tubular wall (12b), the first radial thickness (t₁) is 1.85 mm, the second radial thickness (t₂) is 0.95 mm, and the ratio of the first radial thickness (t₁) to the second radial thickness (t₂) is 1.92. Further, the pitch (P) is 4.8 mm, the curvature radius (R₁) of the inner annular surface (131b) is 1.55 mm, and the curvature radius (R₂) of the outer annular surface (142b) is 1.8 mm. The largest outer diameter (D) and the smallest inner diameter (d) in the first and second tubular sections 150, 160 are similar. That is, the largest outer diameter (D) is 21 mm, the smallest inner diameter (d) is 12.6 mm, and the largest outer radius (r₂) is greater than the smallest inner radius (r₁) by 4.2 mm. The purpose and effect of the first preferred embodiment can be similarly achieved using the fifth preferred embodiment.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. An air supply tube for a nasal breathing apparatus made of a silicone material, and comprises a tubular wall surrounding an axis and defining an axial hole, said tubular wall being corrugated and including a plurality of annularly-shaped concave and convex wall portions alternately arranged along the length of said air supply tube, said convex wall portions being farther from said axis than said concave wall portions, said concave wall portions having a first radial thickness which is larger than a second radial thickness of said convex wall portion.

2. The air supply tube of claim 1, wherein the ratio of said first radial thickness to said second radial thickness is larger than 1, but smaller than 4.

3. The air supply tube of claim 2, wherein each of said concave wall portions has an inner annular surface adjacent to said axial hole, and an outer annular surface opposite to said inner annular surface, each of said convex wall portions has an inner annular surface adjacent to said axial hole and connected between said inner annular surfaces of two adjacent ones of said concave wall portions, and an outer annular surface opposite to said inner annular surface of a respective one of said convex wall portions and connected between said outer annular surfaces of two adjacent ones of said concave wall portions, and wherein a pitch between two adjacent ones of said convex wall portions is in the range of 4˜6 mm.

4. The air supply tube of claim 3, wherein said outer annular surface of each of said convex wall portions has a largest outer diameter, said inner annular surface of each of said concave wall portions has a smallest inner diameter, a ratio of said largest outer diameter to said smallest inner diameter is larger than 1.5, but smaller than 2.

5. The air supply tube of claim 3, wherein said inner annular surface of each of said concave wall portions has a smallest inner radius, and said outer annular surface of each of said convex wall portions has a largest outer radius greater than said smallest inner radius by an amount ranging from 3.5˜4.5 mm.

6. The air supply tube of claim 5, wherein said inner annular surface of each of said concave wall portions further has a curvature radius that is in the range of 3.1˜1.8 mm.

7. The air supply tube of claim 5, wherein said outer annular surface of each of said convex wall portions further has a curvature radius that is in the range of 1.5˜2.2 mm.
